(19) United States
(12) Patent Application Publication
(10) Pub. No.: US 2004/0077037 A1
(43) Pub. Date: Apr. 22, 2004

(54) Method for the selective modification of peptides and proteins

(76) Inventors: Frank Bordusa, Karl-Marx-Platz 2 (DE); Hans-Dieter Jakubke, Albert-Richter-Strasse (DE)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 10/381,838

(22) PCT Filed: Sep. 25, 2001
(86) PCT No.: PCT/EP01/11035

(30) Foreign Application Priority Data
Sep. 27, 2000 (DE) 100 47 857.3

(51) Int. Cl. C12P 21/06

(52) U.S. Cl. 435/68.1; 530/402

(57) ABSTRACT

The invention concerns a method for the regiospecific modification of peptides and proteins with probes and reporter molecules using peptidases in combination with a non-amino acid-like or non-peptide-like substrate mimetic.

The invention concerns a method for the regiospecific modification of peptides and proteins with probes and reporter molecules using biocatalysts.

The sequencing of the genomes of humans and other organisms has resulted in an enormous flood of protein sequences whose biochemical function has not been adequately elucidated in many cases or is not known at all. The proteins are functional gene products and are hence responsible for all the activities of the biological world. For this reason an understanding of protein structure and function is absolutely essential for modern biological research (cf. T. E. Creighton, Proteins structure and Molecular Properties, W.H. Freeman & Co. New York, 1993). Targeted labelling with special probes and reporter groups that do not damage the molecules is essential for such investigations in order to monitor the molecular processes in vitro and in vivo. Main methods are among others fluorescence labelling (cf. R. P. Haugland, Handbook of Fluorescent Probes and Research Chemicals, Molecular Probes, Inc., 1996), the introduction of spin labels (W. L. Hubbel, C. Allenbach, Investigations of Structure and Dynamics in Membrane Proteins Using Site-directed Spin Labeling, Curr. Opin. Struct. Biol. 4 (1994) 566-573), photoaffinity labelling (D. I. Schuster, W. C. Probst, G. K. Ehrling, G. Singh, Photoaffinity labeling, Photochemistry and Photobiology 49 (1988) 785-804) and the “biotin technique” (M. Wilchek, E. A. Bayer, Avidin-Biotin Technology in Meth. Enzymol. V. 184, Academic Press, 1990).

Chemical methods for modifying peptides and proteins have played and still do play an important role in protein research (cf T. Imoto, H. Yamada, Chemical Modification, in Protein Function. A Practical Approach (T. E. Creighton, ed). p. 247-277, IRL Press, 1989; G. E. Means, R. E. Feeney, Chemical Modification of Proteins, Holden-Day, 1971). Hence despite the rapid progress in NMR technology which in the last decade has enabled a complete signal assignment and thus an elucidation of the 3D structures of proteins containing up to 150-200 amino acid building blocks, chemical modification is still used as a tool for determining the spatial structure in solution since the structure of large proteins cannot be analysed by NMR and X-ray structural analysis requires protein crystals which cannot be obtained in very many cases.

Since N-terminal α-amino groups are the preferred targets of selective modifications, the ε amino groups of the ubiquitous lysine residues in proteins and peptides do not allow a targeted introduction of marker and reporter groups or other derivatizations e.g. pegylation at the N-terminus. Chemical acylation reactions are carried out using anhydrides or preferably with active esters such as N-hydroxysuccinimide or 4-nitrophenyl esters which also react with other side chain groups of proteinogenic amino acid residues and thus do not allow a selective N^{α}-modification. Only the phenylacetyl residue has been enzymatically inserted as a protective group for amino acids in peptide syntheses by penicillin acylase in a specificity-determining manner by reversing its natural action (R. Didziapetris, B. Drabnig, V. Schellenberger, H.-D. Jakubke, V. Svedas, FEBS Lett. 287 (1991) 31-33) and has been cleaved by the same enzyme (cf. Review: A. Reidel, H. Waldmann, J. prakt. Chem. 335 (1993) 109-127). Apart from this direct introduction of protective groups, methods have only been described which are based on a transfer of amino acid or peptide derivatives with peptidase-specific amino acid residues in the P₁ position which are already N-terminally labelled by peptidase catalysis and are inevitably not irreversible. In contrast the substrate mimetic concept developed for CN ligations of peptide and protein segments (P. Bordusa, D. Ullmann, C. Elsner, H.-D. Jakubke, Angew. Chem. 109 (1997) 2583-2585; Review: F. Bordusa, Braz. J. Med. Biol. Res. 72 (2000) 469-485) has the advantage of irreversibility.

The object of the invention is the regiospecific biocatalytic modification of peptides and proteins at the N-terminus while excluding secondary reactions as far as possible.

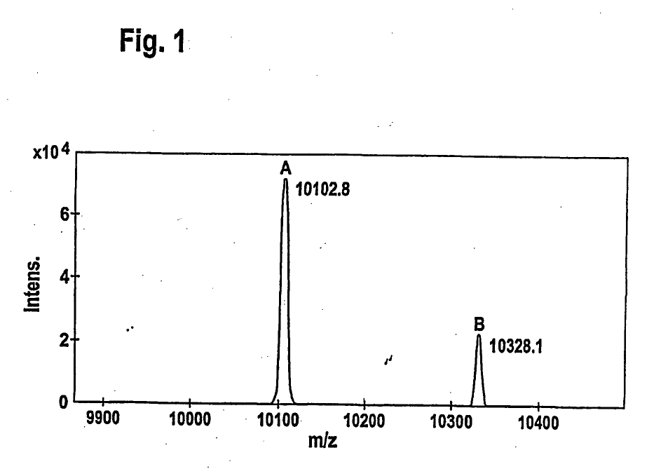

This object is achieved by a method for the selective biocatalytic modification of peptides and/or proteins using a peptidase as a biocatalyst in combination with a non-amino acid-like or non-peptide-like substrate mimetic. The term “substrate mimetic” was coined by Bordusa et al., Angew. Chem. 109 (1997), 2583-2585 and Bordusa et al., Angewandte Chemie International Edition in Englisch, 36 (1997), 2473-2475. The term leaving group is known to a person skilled in the art and is explained by F. Bordusa, Braz. J. Med. Biol. Res. (see above) (in particular in FIG. 1).

According to the present invention the N-terminal biocatalytic modification of a peptide or protein is achieved by using peptidases contrary to the prevailing opinion among experts and in this process the non-amino acid-like or non-peptide-like group to be introduced is specifically manipulated to carry a leaving group in the form of an ester derivative which blocks the native specificity of the enzyme and thus enables the catalysis of an irreversible N^{α}-acylation. The theoretical basis, the postulated reaction mechanism and the preparation of substrate mimetics for various proteases and peptidases is described in the review article by F. Bordusa, Brazilian Journal of Medical and Biological Research 33 (2000), 469-485. In contrast to chemical acylation reactions, the regiospecificity of peptidases has the consequence that reactive side chain functions of trifunctional amino acid building blocks in the peptides and proteins to be modified are not acylated which guarantees an absolutely selective introduction of marker and reporter groups on the N^{α}-amino group of the corresponding peptide or protein. Moreover the group that is to be modified does not already have to be bound to an amino acid or peptide residue to be linked enzymatically which is a prerequisite for methods known in the literature and is prone to reversible cleavage.

The results of the invention are very surprising since, after the biocatalytic introduction of the marker or reporter group according to the invention, the peptidase used for this no longer recognizes such a substituted amide bond as a substrate which thus precludes a reversible enzymatic cleavage. All possible marker or reporter groups can be used as a marker or reporter group such as aminobenzyl, phlorethyl and biotinyl groups. In particular it is possible to use marker groups which are required for the diagnostic use of the peptides or proteins such as haptens (biotin, digoxin, digoxigenin, digitoxin etc.) or labels (dyes, radioactively labelled compounds, fluorescent groups, electrochemiluminescent labels (Elecsys), luminophores etc.). Substances can also be selected as marker or reporter groups which change or improve the properties of proteins such as solubility etc. In particular it is possible to use substances such as polyethylene glycol (PEG) and derivatives thereof to optimize the properties of proteins or peptides such as erythropoietin, insulin, monoclonal antibodies or other therapeutically effective proteins and peptides. Examples of such therapeutic proteins and peptides and substances for optimizing the therapeutic efficacy are known to a person skilled in the art.

Organic-chemical ester derivatives whose acyl residues correspond to the marker or reporter groups that are to be introduced and whose leaving groups carry specificity determinants of selected serine or cysteine peptidases are preferably used for the biocatalytical N^{α}-acylations according to the invention. The terms leaving group and specificity determinants are known to a person skilled in the art (see for example F. Bordusa, Braz. J. Med. Biol. Res. (see above)). As described by F. Bordusa, the leaving group binds a substrate mimetic instead of the specificity-mediating amino acid side chain of the normal substrate (Thormann et al., Biochemistry 38 (1999), 6056-6062). Hence an important property of substrate mimetics is the high affinity of the leaving group for the primary substrate specificity of the respective enzyme e.g. for the strong Glu preference of V8 protease at the S₁ site of the catalytic centre. The discovery, testing and optimization of such leaving groups for substrate mimetics is described by F. Bordusa, Braz. J. Med. Biol. Res. (see above).

The practical procedure i.e. the selection and synthesis of the substrates used for the enzymatic N^{α}-acylation in the form of carboxylic acid esters, the selection of the buffer system, the reaction time etc. is relatively uncritical and can be easily determined by an expert for enzymatic transformations.

According to the invention N^{α}-selective modifications of peptides and proteins are achieved by using a carboxylic acid derivative whose reacting carboxyl function is present as an ester containing a specificity determinant in the leaving group which corresponds to the peptidase that is used, and a peptide or protein to be labelled where the reacting α-amino function is unblocked, in the presence of the appropriate peptidase, in solution at room temperature or also in a frozen state or at low temperatures. Suitable peptidases are for example trypsin, chymotrypsin, V8 protease, Glu-specific endopeptidase from Bacillus licheniformis, subtilisin, mutants of these enzymes such as the trypsin mutant trypsin D189K+K60E (preparation see example 9) or enzymes with similar specificity determinants. In the present description the term peptidase is used in accordance with the nomenclature instead of protease.

If peptide bonds are present in the peptide or protein to be modified which correspond to the specificity of the serine or cysteine peptidases used for the introduction, then either another peptidase with the corresponding specificity determinant in the leaving group of the non-peptidic acyl donor is used which cannot cleave any sensitive peptide bonds in the target sequence or the biocatalytic modification is carried out in a frozen state (cf. Review: M. Hänsler, J.-D. Jakubke, J. Peptide Sci. 2 (1996) 279-289) which prevents undesired proteolytic cleavages as well as high reaction rates.

The modified peptides and proteins can be separated and purified by conventional methods of protein chemistry.

The present invention is elucidated further in the following on the basis of examples.

### EXAMPLE 1

### V8 Protease-Catalysed N-Terminal Introduction of 2-Aminobenzoic Acid Into Peptides

For the model reaction 2-aminobenzoic acid carboxymethylthioester denoted 2-ABz-SCm in the following was used as the carboxy component and the decapeptide Leu-Ala-Leu-Ala-Ser-Ala-Ser-Ala-Phe-Gly was used as the amino component. The 2-ABz-SCm and amino component were used in a ratio of 2:1 at a concentration of 4 mM and 2 mM respectively. An aqueous buffer system containing a small amount of organic solvent was used as the solvent. The reaction was started by adding the enzyme and terminated after almost complete conversion of 2-ABz-SCm by inactivating the enzyme. The reaction was analysed and quantified by chromatographic methods. The enzymatic catalysis led to an almost 99% conversion of Leu-Ala-Leu-Ala-Ser-Ala-Ser-Ala-Phe-Gly into the corresponding N-terminal 2-ABz-modified analogue. The identity of the product of synthesis was checked by conventional methods of organic chemistry.

### EXAMPLE 2

### V8 Protease-Catalysed N-Terminal Introduction of the Phloretyl Group Into Peptides

For the model reaction phloretyl-carboxymethyl thioester denoted phloretyl-SCm in the following was used as the carboxy component and the decapeptide Leu-Ala-Leu-Ala-Lys-Ala-Asp-Ala-Phe-Gly was used as the amino component. The phloretyl-SCm and amino component were used in a ratio of 2:1 at a concentration of 4 mM and 2 mM respectively. An aqueous buffer system containing a small amount of organic solvent was used as the solvent. The reaction was started by adding the enzyme and terminated after almost complete conversion of phloretyl-SCm by inactivating the enzyme. The reaction was analysed and quantified by chromatographic methods. The enzymatic catalysis led to an almost 99.7 % conversion of Leu-Ala-Leu-Ala-Lys-Ala-Asp-Ala-Phe-Gly into the corresponding N-terminal phloretyl-modified analogue. The identity of the product of synthesis was checked by conventional methods of organic chemistry. The reaction resulted neither in an N^{α}-modification of the lysine located in the amino component nor in a detectable proteolytic cleavage after aspartic acid.

### EXAMPLE 3

### α-Chymotrypsin-Catalysed N-Terminal Introduction of 2-Aminobenzoic Acid Into Peptides

For the model reaction 2-aminobenzoic acid-4-guanidinophenyl ester denoted 2-ABz-OGp in the following was used as the carboxy component and the oligopeptide Arg-Ile-Val-Asp-Ala-Val-Ile-Glu-Gln-Val-Lys-Ala-Ala-Gly-Ala-Tyr was used as the amino component. The 2-ABz-OGp and amino component were used in a ratio of 2:1 at a concentration of 4 mM and 2 mM respectively. An aqueous buffer system containing a small amount of organic solvent was used as the solvent. The reaction was started by adding the enzyme and terminated after almost complete conversion of 2-ABz-OGp by inactivating the enzyme. The reaction was analysed and quantified by chromatographic methods. The enzymatic catalysis led to an almost 98.8 % conversion of Arg-Ile-Val-Asp-Ala-Val-Ile-Glu-Gln-Val-Lys-Ala-Ala-Gly-Ala-Tyr into the corresponding N-terminal 2-ABz-modified analogue. The identity of the product of synthesis was checked by conventional methods of organic chemistry.

The reaction resulted neither in a modification of trifunctional side chains, nor in a detectable proteolytic cleavage.

### EXAMPLE 4

### α-Chymotrypsin-Catalysed N-Terminal Introduction of the Phloretyl Group Into Peptides

For the model reaction phloretyl-4-guanidinophenyl ester denoted phloretyl-OGp in the following was used as the carboxy component and the oligopeptide Arg-Ile-Val-Asp-Ala-Val-Ile-Glu-Gln-Val-Lys-Ala-Ala-Gly-Ala-Tyr was used as the amino component. The phloretyl-OGp and amino component were used in a ratio of 2:1 at a concentration of 4 mM and 2 mM respectively. An aqueous buffer system containing a small amount of organic solvent was used as the solvent. The reaction was started by adding the enzyme and terminated after almost complete conversion of phloretyl-OGp by inactivating the enzyme. The reaction was analysed and quantified by chromatographic methods. The enzymatic catalysis led to an almost 99.3 % conversion of Leu-Ala-Leu-Ala-Lys-Ala-Asp-Ala-Phe-Gly into the corresponding N-terminal phloretyl-modified analogue. The identity of the product of synthesis was checked by conventional methods of organic chemistry. The reaction resulted neither in a modification of trifunctional side chains, nor in a detectable proteolytic cleavage.

### EXAMPLE 5

### Trypsin-Catalysed N-Terminal Introduction of 2-Aminobenzoic Acid Into Peptides

For the model reaction 2-aminobenzoic acid-4-guanidinophenyl ester denoted 2-ABzOGp in the following was used as the carboxy component and the decapeptide Leu-Ala-Leu-Ala-Ser-Ala-Ser-Ala-Phe-Gly was used as the amino component. The 2-ABzOGp and amino component were used in a ratio of 2:1 at a concentration of 4 mM and 2 mM respectively. An aqueous buffer system containing a small amount of organic solvent was used as the solvent. The reaction was started by adding the enzyme and terminated after almost complete conversion of 2-ABz-OGp by inactivating the enzyme. The reaction was analysed and quantified by chromatographic methods. The enzymatic catalysis led to an almost 94.4 % conversion of Leu-Ala-Leu-Ala-Ser-Ala-Ser-Ala-Phe-Gly into the corresponding N-terminal 2-ABz-modified analogue. The identity of the product of synthesis was checked by conventional methods of organic chemistry.

### EXAMPLE 6

### Trypsin-Catalysed N-Terminal Introduction of the Phloretyl Group Into Peptides

For the model reaction phloretyl-4-guanidinophenyl ester denoted phloretyl-OGp in the following was used as the carboxy component and the decapeptide Leu-Ala-Leu-Ala-Ser-Ala-Ser-Ala-Phe-Gly was used as the amino component. The phloretyl-OGp and amino component were used in a ratio of 2:1 at a concentration of 4 mM and 2 mM respectively. An aqueous buffer system containing a small amount of organic solvent was used as the solvent. The reaction was started by adding the enzyme and terminated after almost complete conversion of phloretyl-OGp by inactivating the enzyme. The reaction was analysed and quantified by chromatographic methods. The enzymatic catalysis led to a quantitative conversion of Leu-Ala-Leu-Ala-Lys-Ala-Asp-Ala-Phe-Gly into the corresponding N-terminal phloretyl-modified analogue. The identity of the product of synthesis was checked by conventional methods of organic chemistry.

### EXAMPLE 7

### Biotinylation of E. coli Parvulin 10

For the model reaction biotinyl-4-guanidinophenyl ester denoted biotinyl-OGp in the following was used as the carboxyl component and the protein E. coli parvulin 10 was used as the amino component. The biotinyl-OGp and parvulin were used in a ratio of 1:4 at a concentration of 2 mM and 8 mM respectively. An aqueous buffer system containing a small amount of organic solvent was used as the solvent. Specifically 0.1 M HEPES (N₂-[2-hydroxyethyl]piperazine-N′-[2-ethanesulfonic acid] buffer pH 8.0, 0.1 M NaCl, 0.01 M CaCl₂ and 8% (v/v) DMF (dimethylformamide) was used. The reaction was started by adding the enzyme trypsin D189K+K60E (trypsin mutant in which the amino acid D at position 189 is replaced by K or the amino acid K at position 60 is replaced by E; for preparation see example 9) and terminated after a reaction time of two hours. The enzyme was used at a concentration of 6.5×10⁻⁶ M. The reaction was analysed and quantified by MALDI-MS spectroscopy (FIG. 1). The enzymatic catalysis led to a conversion of E. coli parvulin 10 into the N-terminal biotinylated biotinyl-(E. coli parvulin 10).

The primary sequence of E. coli parvulin 10 is known and corresponds to the following amino acid sequence.

AKTAAALHIL VKEEKLALDL LEQIKNGADF GKLAKKHSIC PSGKRGGDLG EFRQGQMVPA FDKVVFSCPV LEPTGPLHTQ FGYHIIKVLY RN

### EXAMPLE 8

### Biotinylation of RNase T1

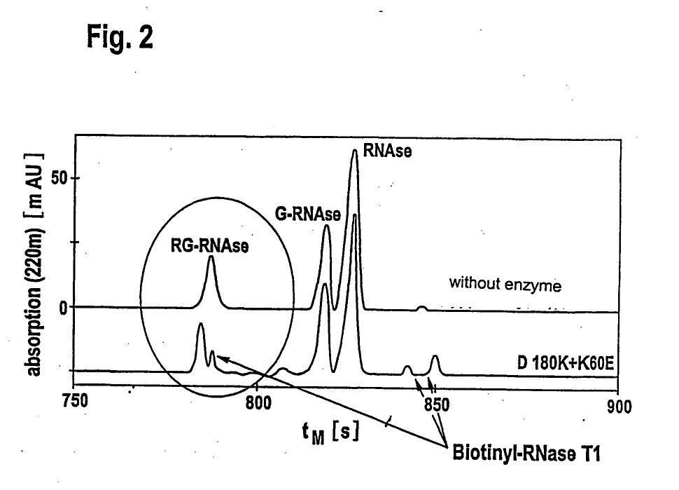

For the model reaction biotinyl-OGp was used as the carboxyl component and the protein RNase T1 was used as the amino component. A variant of RNase T1 was used for the biotinylation with an additional Arg(R)-Gly(G) residue at the N-terminus of the protein. However, due to differences in the in vivo processing of the precursor protein, a species shortened by one amino acid (G-RNase T1) as well as the wild-type (RNase T1) were obtained in addition to the desired RNase T1 variant (RG-RNase T1). This mixture was used without further separation of the individual variants for the enzyme-catalysed biotinylation. The biotinyl-OGp and RNase T1 mixture was used in a ratio of 1:4 at a concentration of 2 mM and 8 mM respectively. An aqueous buffer system as described in example 7 was used as the solvent. The reaction was started by adding the enzyme trypsin D189K+K60E (6.5×10⁻⁶ M). The reaction time was 2 hours. The reaction was analysed and quantified by chromatographic methods. The electropherogram of the capillary electrophoresis is shown in FIG. 2. It can be clearly seen that RG-RNase T1 is almost quantitatively converted into biotinyl-RG-RNase T1.

### EXAMPLE 9

### Preparation of the Trypsin Mutant D189K+K60E

### Plasmids

The E. coli vector pST was used for the site-directed mutagenesis. This contains a part of the Bluescript vector and the gene for anionic rat trypsin which is fused with an afactor leader and with an ADH/GAPDH promoter.

The protein was expressed with the aid of the pYT plasmid, a pBS24 derivative which carries the selection marker for uracil- and leucine-deficient medium.

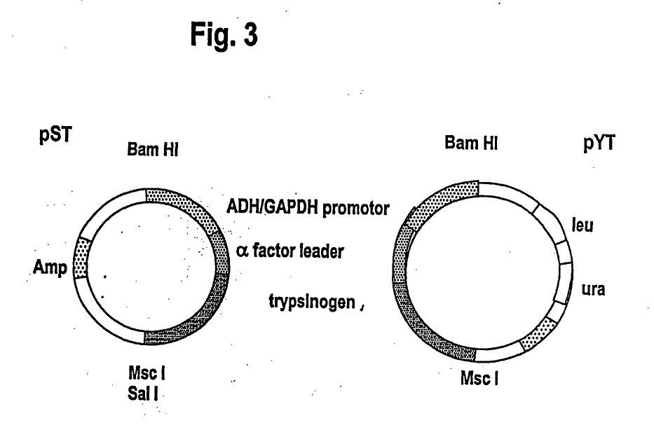

The pST as well as the pYT plasmid have an ampicillin resistance gene. The maps of both vectors i.e. of the plasmids pST (5.4 kb) and pYT (14 kb) with the corresponding cleavage sites are shown in FIG. 3.

### Mutagenesis

Site-directed mutageneses were carried out using the Quik change® kit (STRATAGENE) in the E. coli plasmid pST.

The procedure was like that of a PCR in which both plasmid strands of the pST vector are replicated by PFU polymerase starting with two synthetic oligonucleotide primers which contain the desired mutation. Wild-type pST served as the template to generate individual mutations. These single mutants were in turn the starting point for constructing the double mutants.

The following oligonucleotide primers were used in which the letters in bold type indicate the mutations:

| | | |
|---|---|---|
| D189K | a) | 5′ - GGA GGC AAG AAC GAT TCC TGC - 3′ |
| | b) | 5′ - GCA GGA ATC GTT CTT GCC TCC - 3′ |
| K60E | a) | 5′ - CAC TGC TAT GAG TCC CGC ATC - 3′ |
| | b) | 5′ - GAT GCG GGA CTC ATA GCA GTG - 3′ |

The resulting PCR product was transformed in ultracompetent E. coli XL II blue cells (STRATAGENE). Subsequent selection was carried out on nutrient agar plates containing ampicillin (LB-amp). The picked colonies were transferred to a liquid medium containing ampicillin (LB-amp) and the plasmid was isolated using the SNAP-kit (INVITROGENE) after 1 day of culture. The isolated DNA was checked by electrophoresis using a 1% agarose gel. By sequencing the complete gene it was possible to ensure that only the desired mutations were obtained.

### Subcloning

A subcloning in the pYT expression vector was necessary for all mutants that were generated in the pST plasmid. This was carried out by restriction digestion with Bam HI and Sal I and ligation into the corresponding pYT vector fragment. All vector fragments were applied in the appropriate restriction mixture to a low melting agarose gel (0.8%) and cut out after adequate separation. The gel pieces were melted at 55° C. and pooled according to the desired combination and ligated at 16° C. overnight with T4 DNA ligase. The transformation and plasmid isolation which was again necessary was carried out as described above.

Successful subcloning was tested in the agarose gel by means of a characteristic restriction pattern after double digestion with Eco RI and Bam HI.

By sequencing the complete trypsinogen gene it was possible to ensure that only the desired mutations were obtained.

### Yeast Transformation and Selection

The yeast cell strain that was used is designated Saccharomyces cerevisiae DLM 101α [Mat a,leu 2-3,-112 his 2, 3-11, -15 can 1, ura 3Δ, pep4Δ, [cir⁰], DM 23]. The EZ yeast transformation kit (ZYMO research) was used to prepare competent yeast cells and to transform the pYT plasmids. The selection was carried out on uracil-deficient SC plates by incubation at 30° C. for 3 to 4 days. Leucine-deficient SC plates were inoculated with individual colonies and also incubated for 3 to 4 days at 30° C. which led to an increase in the copy number of the plasmid in the cells. Individual colonies of these plates were used to inoculate precultures of the leucine-deficient SC liquid medium containing 8% glucose. They were incubated by shaking at 30° C. and 120 rpm for 3 days. 20 ml preculture was used as the inoculum to inoculate the 1 litre main cultures containing YPD-medium (1% glucose, 1% bactopeptone, 0.5% yeast extract). The incubation parameters correspond to those of the preculture and they were harvested after 4 days.

### Isolation and Purification of the Trypsin Variants

The cells were firstly separated by centrifuging for 20 min at 4000 rpm and the supernatant was adjusted to pH 4.0 and again centrifuged at 12000 rpm. The almost particle-free supernatant containing trypsinogen was applied to a Toyopearl 650 M (SUPELCO) cation exchanger column equilibrated with 2 mM sodium acetate/100 mM acetic acid (pH 4.5). It was eluted by means of a linear pH gradient starting with 2 mM sodium acetate/100 mM acetic acid (pH 4.5) to 200 mM Tris/HCl (pH 8.0).

The fractions containing trypsinogen were determined and pooled by SDS polyacrylamide gel electrophoresis using a 15% polyacrylamide gel. The volumes of the protein solutions were concentrated to about 10 to 15 ml by means of Centriprep concentrators (AMICON).

Activation of the trypsinogen variant to the corresponding trypsin D189K+K60E was carried out using highly purified enterokinase (BIOZYME) at pH 6.5 and was monitored by SDS gel electrophoresis.

The activated enzyme was purified using a Biocad Sprint perfusion chromatography system (PERSEPTIVE BIOSYSTEMS). The protein samples were separated on a 5% Bis/Tris propane pH 6.0 equilibrated POROS 20 HQ—anion exchanger column (4×100 mm, PERSEPTIVE BIOSYSTEMS) and subsequent gradient elution up to 95% 3 M NaCl solution. The fractions containing trypsin were examined with the aid of an SDS gel and pooled. They were subsequently dialysed against 1 mM HCl at 4° C. and the samples were concentrated with Centriprep concentrators to 2 to 4 ml.

The final yields were about 2 to 5 mg protein per litre culture medium.

### Determination of the Concentration

The protein concentration of the preparations was determined according to the method of Bradford on a spectrophotometer at a wavelength of 595 nm. The calibration curve was plotted on the basis of a serial dilution of bovine trypsin between 50 μm/ml and 1 mg/ml.

1. Method for the biocatalytic modification of peptides and proteins characterized in that
a peptidase in combination with a non-amino acid-like or non-peptide-like substrate mimetic is used as the biocatalyst.

2. Method as claimed in claim 1, characterized in that
a carboxylic acid ester is used as the acylation component whose acyl moiety corresponds to the modification group and whose leaving group contains the specificity determinants of the peptidase used for the catalysis.

3. Method as claimed in claim 1 or 2, characterized in that
the reaction is carried out in an aqueous medium at room temperature or in a frozen aqueous system or at low temperatures between −5 and −20° C.

4. Method as claimed in one of the previous claims, characterized in that trypsin, chymotrypsin, V8 protease, glu-specific endopeptidase from Bacillus licheniformis, subtilisin among others or mutants of these enzymes or enzymes with similar specificity determinants are used as the peptidases.

5. Use of peptidases, characterized in that
they are used to biocatalytically introduce marker and reporter groups into peptides and proteins.

6. Use of peptidases as claimed in claim 5, characterized in that they are used for the biocatalytic introduction of marker and reporter groups into peptides and proteins while avoiding their reversible enzymatic cleavage by the fact that the marker or reporter group that is introduced carries an ester derivative as the leaving group which blocks the native specificity of the enzyme that is used.

7. Use of peptidases as claimed in claim 5 and 6, characterized in that
trypsin, chymotrypsin, V8 protease, glu-specific endopeptidase from Bacillus licheniformis, subtilisin or mutants of these enzymes or enzymes with similar specificity determinants are used as the peptidases.
